(19) United States
(12) Patent Application Publication
(10) Pub. No.: US 2009/0125078 A1
(43) Pub. Date: May 14, 2009

(54) SELECTING CARDIAC PACING SITES

(75) Inventors: Daniel R. Kaiser, Plymouth, MN (US); Michael R. Neidert, Salthill, CO (US); Nicholas D. Skadsberg, Blaine, MN (US); Kenneth G. Gardeski, Plymouth, MN (US); Lawrence J. Mulligan, Andover, MN (US); James F. Kelley, Coon Rapids, MN (US); Michael B. Shelton, Minneapolis, MN (US); Trent M. Fischer, St. Paul, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: MEDTRONIC, INC., Minneapolis, MN (US)

(21) Appl. No.: 12/245,570

(22) Filed: Oct. 3, 2008

Related U.S. Application Data
(60) Provisional application No. 60/977,098, filed on Oct. 3, 2007.

(51) Int. Cl. A61N 1/37

(52) U.S. Cl. 607/27

(57) ABSTRACT

A method for selecting a cardiac pacing site includes steps of: securing first and second electromagnetic receiver coils at first and second positions, respectively, along a heart wall; collecting a set of non-paced heart wall motion data from each of the coils secured at the corresponding positions; applying cardiac pacing stimulation at at least one first pacing site; collecting a first set of paced heart wall motion data from each of the secured coils; comparing the non-paced heart wall motion data to the first set of paced heart wall motion data; and determining, based on the comparing, whether to maintain pacing at the at least one first cardiac pacing site or to apply pacing stimulation at a second pacing site for collection of a second set of paced heart wall motion data. The at least one first pacing site may include a right ventricular site and a left ventricular site.

### CROSS-REFERENCE TO RELATED APPLICATION

The present application claims priority and other benefits from U.S. Provisional Patent Application Ser. No. 60/977,098, which was filed on Oct. 3, 2007, and which is incorporated herein by reference in its entirety.

### TECHNICAL FIELD

The present disclosure pertains to cardiac pacing and more particularly to methods for selecting cardiac pacing sites.

### BACKGROUND

In recent years cardiac resynchronization therapy (CRT) for patients suffering from chronic heart failure has been shown to increase exercise capacity and a quality of life for these patients. CRT is typically administered via bi-ventricular pacing delivered via implanted medical electrodes, and the outcome of the therapy is often highly dependent upon selecting, and then successfully implanting the electrodes at appropriate pacing sites. In this context, as well as others, for example, physiological or dual chamber pacing, alternative pacing sites may be evaluated via measures of the electrical and/or mechanical response of the heart to the pacing. Many assert that pacing is most effective if mechanical synchrony between the right and left ventricle can be maintained or re-established, thus many physicians prefer to assess a mechanical, or hemodynamic, response of the heart to pacing at various implant sites before selecting one or more locations for chronic pacing. Tissue Doppler Imaging (TDI) is one of several methods currently employed to assess the mechanical response of a heart to pacing, but there is still a need for methods that can simplify intra-operative monitoring of the mechanical response of the heart to pacing at various sites, for example, to facilitate selection of effective bi-ventricular pacing sites.

### BRIEF DESCRIPTION OF THE DRAWINGS

The following drawings are illustrative of particular embodiments of the present disclosure and therefore do not limit the scope of the disclosure. The drawings are not to scale (unless so stated) and are intended for use in conjunction with the explanations in the following detailed description. Embodiments of the present disclosure will hereinafter be described in conjunction with the appended drawings, wherein like numerals denote like elements.

FIG. 1 is a diagram of an exemplary system for carrying out methods of the present disclosure.

FIGS. 2A-C are schematics showing various cardiac monitoring and pacing sites according to some methods of the present disclosure.

FIG. 3 is a plan view of a distal portion of a lead employed by some methods of the present disclosure.

FIGS. 4A-C are exemplary analysis plots which may be generated with data collected by some methods of the present disclosure.

### DETAILED DESCRIPTION

The following detailed description is exemplary in nature and is not intended to limit the scope, applicability, or configuration of the disclosure in any way. Rather, the following description provides practical illustrations for implementing exemplary embodiments of the present disclosure. Constructions, materials, dimensions, and manufacturing processes suitable for making embodiments of the present are known to those of skill in the field of the disclosure.

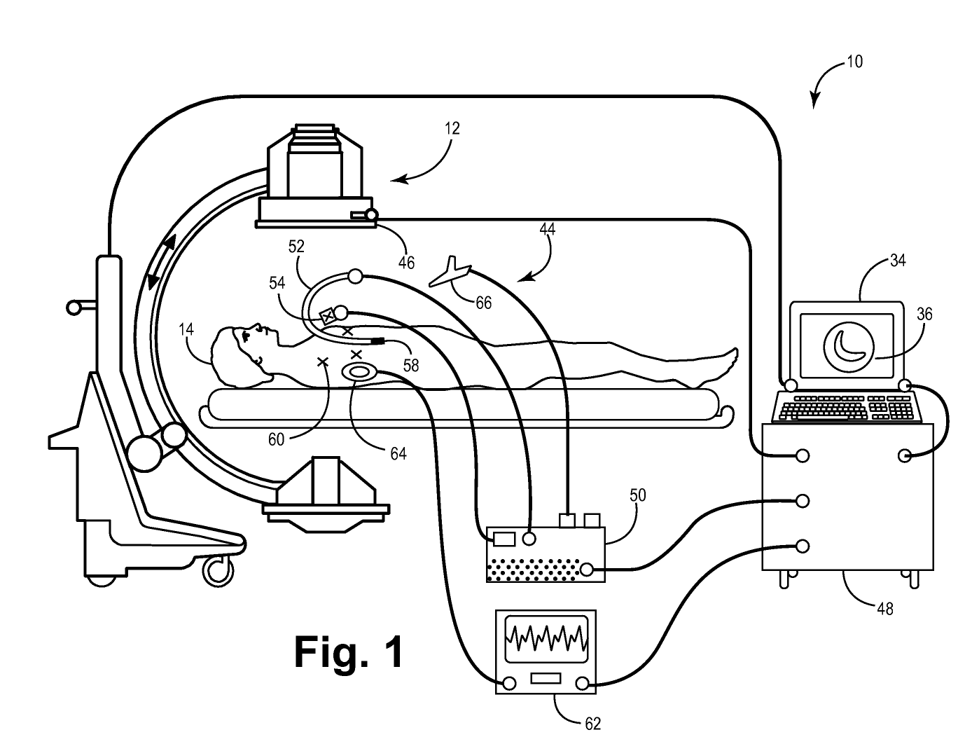

In parallel with the development of CRT, techniques employing image-guided surgical navigation technology have been developed for the navigation of catheters, or leads, within the heart in order to assist in the placement of pacing electrodes. A particular image-guided navigation system, described in co-pending and commonly assigned U.S. patent application 2004/0097806 entitled NAVIGATION SYSTEM FOR CARDIAC THERAPIES, which is hereby incorporated by reference in its entirety, may be employed, by methods of the present disclosure, for the monitoring of cardiac wall motion in response to pacing at various sites. FIG. 1, which has been borrowed from the aforementioned patent application, is a diagram of the system 10. It should be noted that the principles described herein may be applied in alternative contexts in which medical electrical leads are employed.

FIG. 1 illustrates system 10 including a fluoroscopic C-arm imaging device 12, an electromagnetic navigation or tracking device 44, a gating device or electrocardiograph 62, and a controller or work station 34, which receives input from each of the aforementioned devices. Tracking device 44 includes a transmitter coil array 46, which is controlled, or driven, by a coil array controller 48. Coil array controller 48 may drive each coil, in transmitter coil array 46, in a time division multiplex or a frequency division multiplex manner. In this regard, each coil may be driven separately, at a distinct time, or all of the coils may be driven simultaneously, wherein each is driven at a different frequency. Thus, coil array controller 48 drives coils in array 46 in order to generate electromagnetic fields, within a patient 14, in the area where the medical procedure is being performed, which is sometimes referred to as the patient space. The electromagnetic fields, generated within the patient space, induce currents in at least one localization sensor 58, for example, an electromagnetic receiver coil, which is coupled to a lead or catheter 52, as is further discussed herein. These induced currents, or signals, are delivered from catheter 52 to a navigation probe interface 50, which provides the necessary electrical isolation for navigation system 10. Probe interface 50 further includes amplifiers, filters and buffers required to directly interface with sensor(s) 58 of catheter 52. Catheter 52 may employ a wireless communications channel, as opposed to being directly coupled to probe interface 50.

Tracking device 44 functions to transfer the signals to coil array controller 48, which then processes the signals in order to generate, and superimpose, an icon, which represents the location of the catheter, onto images generated by imaging device 12, which are displayed on a display 36 of workstation 34. Electrocardiograph 62 provides for a time-gated acquisition of the signals from coil 58 and/or the images from imaging device 12, for example, by triggering acquisition off of a measured R-wave, or ventricular depolarization, which may be sensed by skin electrodes 64, which are coupled to electrocardiograph 62. FIG. 1 further illustrates tracking device 44 including a dynamic reference frame 54, which is fixed to patient 14 to track movement of patient 14 for registration correlation in order to maintain accurate information concerning the catheter location. Patient registration may be accomplished by selecting and storing particular points or landmarks 60 in memory, from pre-acquired images and then by touching the corresponding points on a patient's anatomy with a pointer probe 66. A landmark is an anatomical feature that is generally common to all patients. A complete and detailed description of system 10 can be found in the aforementioned '806 application, which has been incorporated by reference.

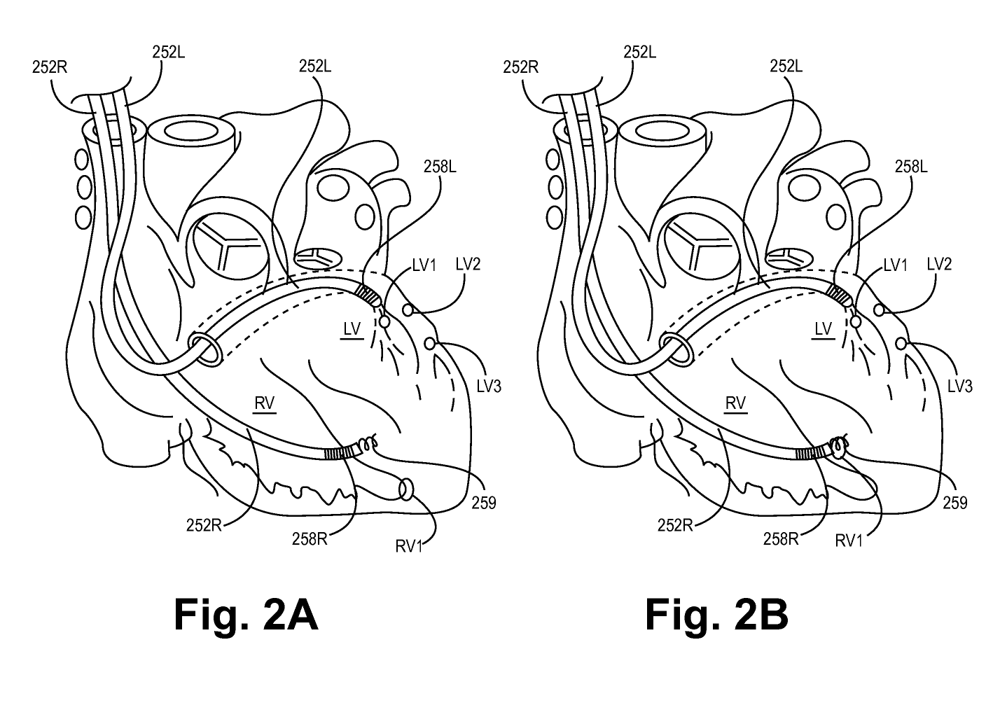

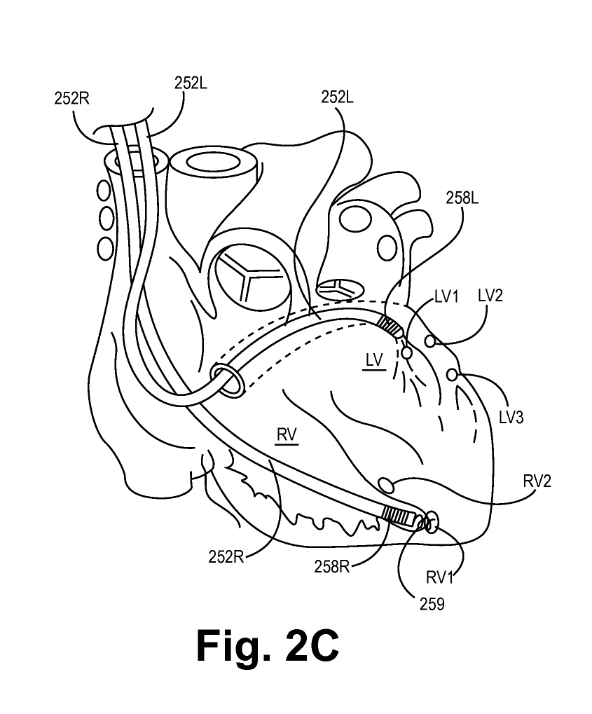

According to embodiments of the present disclosure, a system, similar to system 10, includes at least one pair of electromagnetic receiver coils utilized not only in a navigational capacity, as described in the '806 application, but also in a monitoring capacity for the purpose of selecting one or more cardiac pacing sites intra-operatively, that is, at a time of pacing electrode implant. FIGS. 2A-C are schematics showing various cardiac monitoring and pacing sites according to some methods of the present disclosure. FIGS. 2A-C illustrate a first elongate lead 252R extending into a right ventricle (RV) and a second elongate lead 252L extending into a coronary vein over a surface of a left ventricle (LV); each of leads 252R and 252L include an electromagnetic receiver coil 258R, 258L, respectively, which has been positioned to monitor cardiac wall motion. Voltage signals from coils 258L, 258R, which are generated by a current induced therein by an external magnetic field, for example, created by coil array controller 48 driving coils in array 46 (FIG. 1), facilitate creation of a virtual representation of leads 252R, 252L, respectively, in proximity to the RV and LV walls, and thereby provide RV and LV heart wall motion data. (The term ‘lead’ is employed in a generic sense to denote a body carrying at least one receiver coil and an associated lead wire; as such, either or both of leads 252R and 252L may further be adapted to carry out addition functions, for example, in facilitating delivery of a pacing electrode to a target site, and can, thus, in various embodiments, take the form of a guidewire or catheter.) It should be noted that the voltage signals from each of coils 258R, 258L may be used for image guided navigation of leads 252R and 252L, respectively, to the illustrated positions, for example, according to methods described in the aforementioned '806 application. Furthermore, it should be noted, that each of leads 252R, 252L may include a plurality of receiver coils spaced apart from one another along a length thereof, in order to provide more enhanced wall motion data.

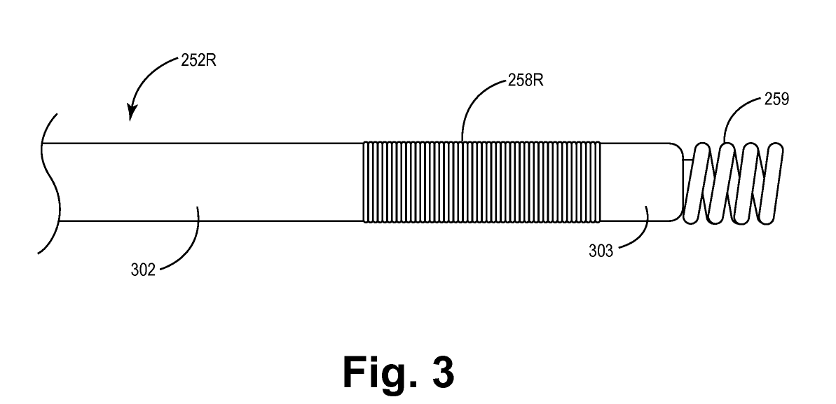

FIG. 3 is a plan view of a distal portion of lead 252R, according to some embodiments of the present disclosure. FIG. 3 illustrates a fixation element 259 terminating a distal segment 303 of lead 252R, coil 258R extending proximally from segment 303, and a body 302 of lead 252R extending proximally from coil 258R; element 259 serves to secure coil 258R at a position along a heart wall. According to preferred embodiments of the present disclosure, segment 303 is relatively rigid, for example, being formed from a 75D durometer polyurethane, so that coil 258R will move in sync with that portion of the heart wall to which element 259 is fixed, while body 302 is relatively supple, or flexible, for example, being formed predominately from silicone rubber, so as not to influence the response of coil 258R to the wall motion. Those skilled in the art will appreciate that lead wires for coil 258R extend proximally therefrom, within body 302 to couple, for example, with probe interface 50 (FIG. 1); an exemplary assembly for coil 258R (as well as for coil 258L), which may be incorporated by embodiments of the present disclosure, is described in conjunction with FIGS. 3A-C of a commonly assigned and co-pending patent application entitled THERAPY DELIVERY SYSTEM INCLUDING A NAVIGATION ELEMENT and having the Ser. No. 11/322,393 (Atty. Docket no. P-20898.00), and the FIGS. 3A-C, along with the associated description, of this application are hereby incorporated by reference. It should be noted that, in the context of the present disclosure, fixation of a receiver coil, for example, coil 258L, to a heart wall can encompass fixation to a coronary vein. Furthermore, it should be noted that methods of the present disclosure may alternately be carried out by leadless, or wireless, electromagnetic receiver coils, an example of which is described in co-pending and commonly-assigned patent application Ser. No. 11/565,283 (Atty. Docket no. P-22326.00), which is hereby incorporated by reference in its entirety.

With reference back to FIGS. 2A-B, according to some methods of the present disclosure, coil 258R is fixed, or secured, at a position along the RV septal wall by fixation element 259 of lead 252R, and coil 258L has been secured along the LV wall by lodging a distal tip of lead 252L deep within the coronary vein. It should be noted that lead 252L may also include a fixation element to secure coil 258R at a position along the LV wall, so that the secured position is not dependent upon an anatomy of the coronary vasculature. An alternate position for the fixation of coil 258R, which is in closer proximity to the RV apex, is shown in FIG. 2C. It should be noted that, although FIGS. 2A-C illustrate transvenous approaches for positioning coils 258R, 258L, within the venous system, the disclosure is not so limited, and one or both of coils 258R, 258L may be fixed, or secured to an epicardial surface of the heart, for example, via a trans-thoracic or sub-xiphoid approach known to those skilled in the art.

With further reference to FIGS. 2A-C, non-paced heart wall motion data may be collected, or sampled, using conventional techniques, from coils 258R, 258L for comparison with sets of paced heart wall motion data that result from pacing at an RV site RV1 (FIG. 2A) in combination with pacing at different LV sites LV1, LV2, LV3. Alternately, or additionally, sets of paced heart wall motion data that result from pacing at another RV site RV2 (FIG. 2B) in combination with pacing at the LV sites LV1, LV2, LV3 may be compared to the non-paced heart wall motion data. According to one method, heart wall motion data sets, for example, averaged over five heart beats, for the non-paced condition and each of the paced conditions that correspond to each pair of selected pacing sites, may be collected and stored for projection onto a pre-acquired image of the patient's heart, for example, a fluoroscopic image generated by imaging device 12 (FIG. 1). Each of these wall motion data sets, which are presented by the motion of the virtual representation of receiver coil 258R on the pre-acquired image, may then be viewed, for example, on display 36 of workstation 34 (FIG. 1), when a user ‘clicks on’, or selects via an interface of workstation 34, landmarks in the pre-acquired image that have been associated with each of the selected pacing sites.

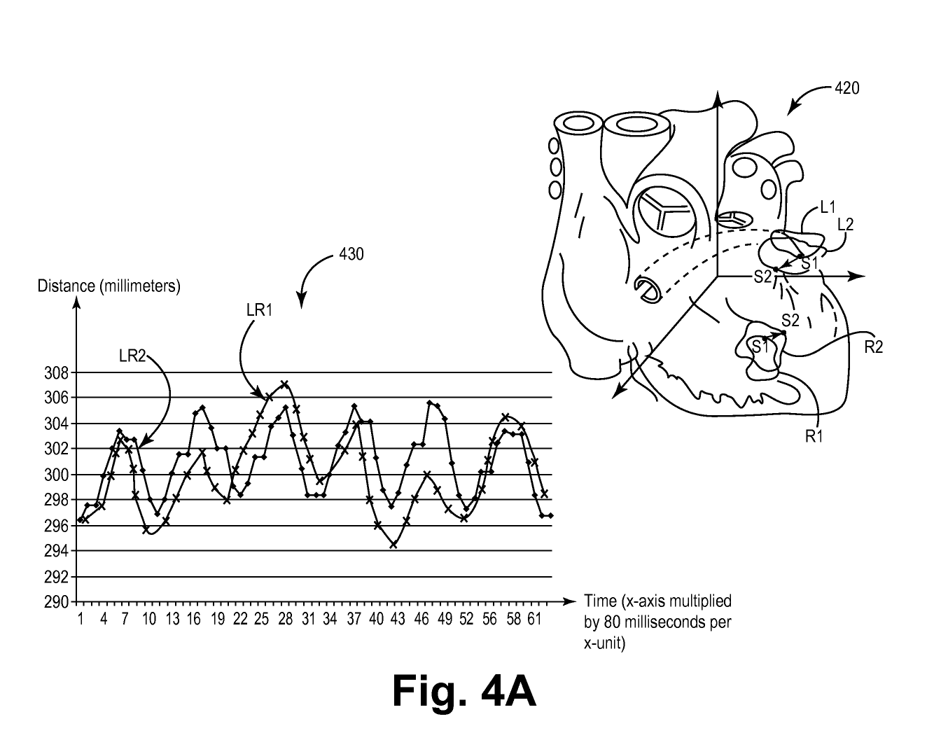

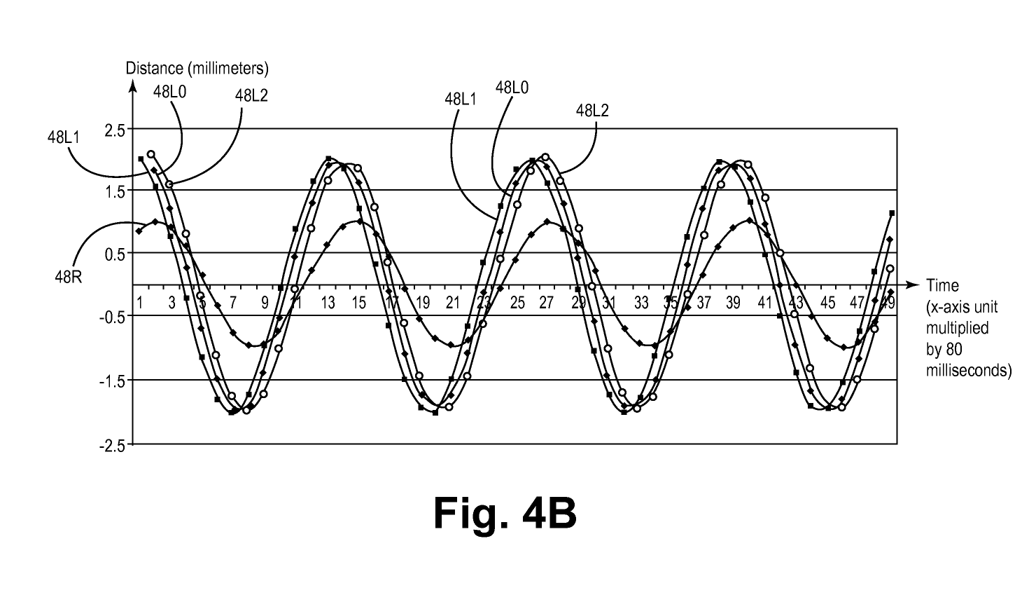

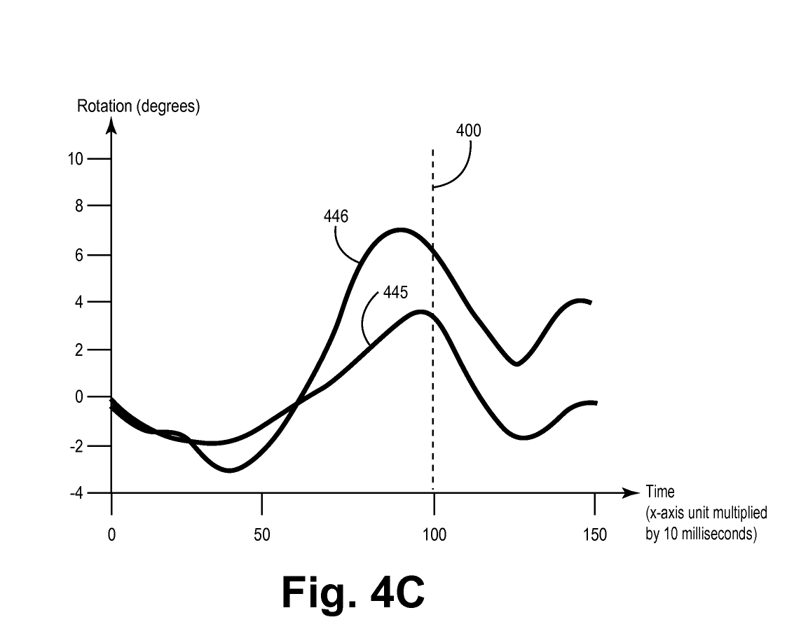

FIG. 4A is an exemplary display including a three dimensional plot 420 of wall motion data, for example, averaged over six cycles, which is superimposed on an image of a patient's heart, and a two dimensional plot 430, of distances mapped between coils 258R, 258L, at particular points in time for each of the six cycles. The plotted wall motion data is not actual data, but is representative of data that could be collected from coils 258R, 258L. Plot 420 shows a first condition represented by a pair of simultaneous motion loops L1 and R1 created, for example, from averaged wall motion data collected from coils 258L and 258R, respectively, either when the heart is not paced, or when the heart is paced at at least one of pacing sites LV1, LV2, or LV3. For comparison, plot 420 also shows a second condition, represented by a pair of simultaneous motion loops L2 and R2 created, for example, from averaged wall motion data collected from coils 258L and 258R, for pacing that has been adjusted, either being applied (vs. no pacing), or being applied at a different site, from that which resulted in loops L1 and R1. Point S1 on each of loops L1 and R1 corresponds to an approximate position of the respective heart wall portion at systole for the first condition, and point S2 on each of loops L2, R2 to an approximate position of the respective heart wall portion at systole for the second condition. With reference to points S1, S2, it may be appreciated that motion loops L2, R2 show a greater contraction between the heart wall portions and a greater relative rotation therebetween, which is indicative of a twisting, or torsion, from apex to base, that will be described in greater detail below. Plot 430 presents the first and second conditions in a different manner wherein a distance between corresponding points of each of the motion loops that have been averaged to create loops L1 and R1, are plotted over time for the six cycles for comparison with a distance between corresponding points of each of the motion loops that have been averaged to create loops L2 and R2. The six cycles may be identified by the six peak magnitudes for each curve. Distances between points of loop L1 and points of loop R1 make up curve LR1, and distances between points of loop L2 and points of loop R2 make up curve LR2. With reference to plot 430 it may be appreciated that the repeatability of magnitudes of the distances making up curve LR2 is greater than that for curve LR1 over the six cycles, which may be an indication of better synchrony between left and right heart wall motion. Thus, with reference to the display of FIG. 4A, one may determine that the pacing resulting in the second condition, represented by loops L2, R2 and curve LR2, provides a better hemodynamic response than the lack of pacing or pacing at another site resulting in the first condition, represented by loops L1, R1 and curve LR1. Other methods for comparing heart wall motion data will be discussed below, in conjunction with FIGS. 4B-C.

Pacing may be applied at the sites, either endocardial or epicardial, by pacing lead electrodes which have been delivered to the sites by a transvenous or a trans-thoracic or a sub-xiphoid approach, according to a variety of methods well known to those skilled in the art. According to some embodiments of the present disclosure, one or both of leads 252R, 252L further include an electrode for delivering the pacing stimulation; for example, in FIG. 2B fixation element 259 may double as a pacing electrode to deliver pacing stimulation at site RV2. According to methods of the present disclosure, wall motion data for any group of pacing sites may be iteratively collected for comparison with non-paced wall motion data, in order to select one or more preferred pacing sites.

The pacing sites shown are in areas generally corresponding to effective bi-ventricular pacing sites, but, it should be noted that methods of the present disclosure are not limited to these particular pacing sites. In the context of bi-ventricular pacing for CRT, a difference between paced and non-paced heart wall motion is typically sought, since non-paced wall motion will be asynchronous and the objective is to achieve synchrony; however in a different context, for example, in selecting one or more pacing sites for bradycardia or tachyarrhythmia therapy, a similarity between paced and non-paced heart wall motion is sought, since the objective is to maintain the already synchronous heart wall motion.

According to some methods, the wall motion data corresponding to various pacing sites from secured RV and LV coils, for example, coils 258R and 258L, respectively, is processed and plotted to provide a picture of RV and LV wall motion with respect to one another, in the time domain. FIG. 4B is an exemplary plot of a net motion of three-dimensional wall motion data. The plotted wall motion data is not actual data, but is representative of data that could be collected from coils 258R, 258L. With reference to FIG. 4B, in conjunction with FIG. 2A, a first curve 48R is generated from non-paced wall motion data collected from coil 258R, a second curve 48L0 is generated from non-paced wall motion data collected from coil 258L, a third curve 48L1 is generated from paced wall motion data collected from coil 258L, wherein pacing is applied at a first pair of sites, RV1 and LV1, and a fourth curve 48L2 is generated from paced wall motion data collected from coil 258L, wherein pacing is applied at a second pair of sites, RV1 and LV2. The plot of FIG. 4B indicates that pacing at sites RV1 and LV2, which results in the wall motion depicted by curve 48L2, brings LV heart wall motion closer into phase, or synchrony with RV heart wall motion, which is represented by first curve 48R.

According to some other methods, preferred pacing sites may be selected according to maximum cardiac wall motion, either RV, LV or both. According to an exemplary method of this type, the wall motion data from secured coils 258R, 258L, positioned as shown in FIG. 2C, is processed to generate a plot describing a differential rotation between an apex and a base of the heart. Alternately, wall motion data from a plurality of receiver coils disposed along a length of lead 252R positioned in the RV as shown in FIG. 2C and from a plurality of receiver coils disposed along a length of lead 252L positioned in the cardiac vein, as shown in FIG. 2C, can provide more detailed information concerning the differential rotation. This differential rotation is indicative of the characteristic twisting or torsion, from apex to base, of cardiac contraction; the twisting is commonly described as a wringing-out motion that ‘squeezes’ the blood out from the RV and LV during systole. The effectiveness of the motion is often measured in terms of an ejection fraction, that is, a ratio of the blood that is ejected from the LV to that which is contained in the LV at the peak of filling, or diastole. FIG. 4C is a plot of relative rotation (ordinate) between apex and base, in terms of degrees, versus time (abscissa), in terms of percent of systole, which may be generated from a torsion analysis of the wall motion data for a paced and an un-paced condition. Dashed line 400 corresponds to a closing of the aortic valve at 100% systole. A first curve 445 of the plot is indicative of a relatively low ejection fraction, and may correspond to an un-paced condition, while a second curve 446 is indicative of a more normal ejection fraction, wherein the relative rotation between apex and base has been increased, for example, via pacing. One or more additional pacing sites may be tested, and the corresponding sets of wall motion data collected and plotted, per FIG. 4C, to find out if an even greater relative rotation can be induced. According to another exemplary method, wall motion indicative of ejection fraction may be observed in terms of short and/or long axis contraction and expansion for the LV.

With reference back to FIG. 1, pre-programmed algorithms of workstation 34 may process wall motion data collected from coils 258R, 258L to generate plots, for example, like those described above in conjunction with FIGS. 4A-C. Such plots, for example, displayed on display 36 of workstation 34, can help a physician to select one or more effective pacing sites by facilitating a methodical comparison between baseline non-paced mechanical function of the heart and the mechanical function thereof in response to pacing at various sites.

In the foregoing detailed description, the disclosure has been described with reference to specific embodiments. However, it may be appreciated that various modifications and changes can be made without departing from the scope of the disclosure as set forth in the appended claims.

We claim:

1. A method for selecting at least one cardiac pacing site, the method comprising:
directing a first elongate lead to a position along a portion of a right ventricular heart wall, the first lead including an electromagnetic receiver coil;
securing the electromagnetic receiver coil of the first lead at the position along the right ventricular heart wall;
inducing a signal in the electromagnetic receiver coil of the first lead by generating a magnetic field, the signal facilitating creation of a virtual representation of a portion of the first lead to direct the first lead to the first position, and to track wall motion at the position along the right ventricular heart wall;
directing a second elongate lead to a position along a left ventricular heart wall, the second lead including an electromagnetic receiver coil;
securing the electromagnetic receiver coil of the second lead at the position along the left ventricular heart wall;
inducing a signal in the electromagnetic receiver coil of the second lead by generating a magnetic field, the signal facilitating creation of a virtual representation of a portion of the second lead, in order to direct the second lead to the second position, and to track wall motion at the position along the left ventricular heart wall;
collecting a set of non-paced heart wall motion data from the signal of each of the electromagnetic receiver coils secured at the corresponding position;
applying cardiac pacing stimulation at an at least one first cardiac pacing site;
collecting a first set of paced heart wall motion data from the signal of each of the electromagnetic receiver coils secured at the corresponding position;
comparing the set of non-paced heart wall motion data to the first set of paced heart wall motion data; and
determining, based on the comparing, whether to maintain pacing at the at least one first cardiac pacing site or to apply pacing stimulation at a second cardiac pacing site for collection of a second set of paced heart wall motion data.

2. The method of claim 1, wherein the portion of the right ventricular heart wall comprises a septal portion.

3. The method of claim 1, wherein the portion of the right ventricular heart wall comprises a portion located in proximity to an apex of the heart.

4. The method of claim 1, wherein the portion of the left ventricular heart wall comprises a portion in proximity to a base of the heart.

5. The method of claim 1, wherein directing the first elongate lead comprises directing, transvenously, to the position along the left ventricular heart wall, through a cardiac vein.

6. The method of claim 1, wherein directing the first elongate lead comprises directing, trans-thoracic, to the position along the left ventricular heart wall.

7. The method of claim 1, wherein securing at least one of the electromagnetic receiver coils comprises engaging a fixation element of the corresponding lead to the corresponding heart wall.

8. The method of claim 1, wherein securing the electromagnetic receiver coil of the second lead at the position along the left ventricular heart wall comprises lodging the at least one lead in a cardiac vein.

9. The method of claim 1, wherein comparing comprises a time domain analysis to determine a degree of synchrony between the wall motion at the position along the right ventricle and the wall motion at the position along the left ventricle.

10. The method of claim 1, wherein comparing comprises a torsional analysis to determine a relative rotation between the position along the right ventricular wall and the position along the left ventricular wall.

11. The method of claim 1, wherein the at least one first cardiac pacing site comprises a left ventricular pacing site and a right ventricular pacing site.

12. The method of claim 11, wherein the first elongate lead further includes an electrode, employed for applying the pacing stimulation to the right ventricular pacing site.

13. The method of claim 11, wherein the second elongate lead further includes an electrode, employed for applying the pacing stimulation to the left ventricular pacing site.

14. The method of claim 11, wherein the left ventricular pacing site comprises a site in proximity to a base of the heart.

15. The method of claim 11, wherein the right ventricular pacing site comprises a site located in proximity to an apex of the heart.

16. The method of claim 11, wherein the second cardiac pacing site comprises one of: another left ventricular pacing site and another right ventricular pacing site.

17. The method of claim 1, further comprising:
applying cardiac pacing stimulation at an at least one second cardiac pacing site;
collecting a second set of paced heart wall motion data from the signal of each of the electromagnetic receiver coils secured at the corresponding position;
comparing the set of non-paced heart wall motion data to the second set of paced heart wall motion data; and
determining, based on the comparing, whether to maintain pacing at the at least one first pacing site, or at the at least one second pacing site, or to apply pacing stimulation at a third pacing site for collection of a third set of paced heart wall motion data.

18. A method for selecting at least one cardiac pacing site, the method comprising:
directing a first elongate lead to a first position along a heart wall, the first lead including an electromagnetic receiver coil and the first position being located in proximity to a base of the heart;
securing the electromagnetic receiver coil of the first lead at the first position, the electromagnetic receiver coil producing a signal in response to an externally induced magnetic field, the signal facilitating creation of a virtual representation of a portion of the first lead to direct the first lead to the first position, and to track wall motion at the first position;
directing a second elongate lead to a second position along the heart wall, the second lead including an electromagnetic receiver coil and the second position being located in proximity to an apex of the heart;
securing the at least one electromagnetic receiver coil of the second lead at the second position, the electromagnetic receiver coil producing a signal in response to an externally induced magnetic field, the signal facilitating creation of a virtual representation of a portion of the second lead to direct the second lead to the second position, and to track wall motion at the second position;
collecting a set of non-paced heart wall motion data from the signal of each of the electromagnetic receiver coils secured at the corresponding position;
applying cardiac pacing stimulation at an at least one first cardiac pacing site;
collecting a first set of paced heart wall motion data from the signal of each of the electromagnetic receiver coils secured at the corresponding position;
comparing the set of non-paced heart wall motion data to the first set of paced heart wall motion data; and
determining, based on the comparing, whether to maintain pacing at the at least one first cardiac pacing site or to apply pacing stimulation at a second cardiac pacing site for collection of a second set of paced heart wall motion data.

19. The method of claim 18, wherein securing at least one of the receiver coils comprises engaging a fixation element of the corresponding lead to the heart wall.

20. The method of claim 18, wherein comparing comprises a torsional analysis to determine a relative rotation between the first position and the second position.

21. The method of claim 18, wherein the at least one first cardiac pacing site comprises a left ventricular pacing site and a right ventricular pacing site.

22. The method of claim 21, wherein the second cardiac pacing site comprises one of: another left ventricular pacing site and another right ventricular pacing site.

23. A method for selecting at least one cardiac pacing site, the method comprising:
securing a first electromagnetic receiver coil at a first position along a heart wall, the first electromagnetic receiver coil producing a signal in response to an externally induced magnetic field, the signal facilitating creation of a virtual representation of the first receiver coil to track wall motion at the first position;
securing a second electromagnetic receiver coil at a second position along the heart wall, the second electromagnetic receiver coil producing a signal in response to an externally induced magnetic field, the signal facilitating creation of a virtual representation of the second receiver coil to track wall motion at the second position along the heart wall;
collecting a set of non-paced heart wall motion data from the signal of each of the first and second electromagnetic receiver coils secured at the corresponding position;
applying cardiac pacing stimulation at an at least one first cardiac pacing site;
collecting a first set of paced heart wall motion data from the signal of each of the electromagnetic receiver coils secured at the corresponding position;
comparing the set of non-paced heart wall motion data to the first set of paced heart wall motion data; and
determining, based on the comparing, whether to maintain pacing at the at least one first cardiac pacing site or to apply pacing stimulation at an at least one second cardiac pacing site for collection of a second set of paced heart wall motion data.

24. The method of claim 23, wherein at least one of the first and second positions comprises a position located along a right ventricular septum.

25. The method of claim 23, wherein at least one of the first and second positions comprises a position located in proximity to an apex of the heart.

26. The method of claim 23, wherein at least one of the first and second positions comprises a positions located in proximity to a base of the heart.

27. The method of claim 23, wherein at least one of the first and second positions comprises a position located on a left ventricular wall.

28. The method of claim 23, wherein:
the first position comprises a position located along a right ventricular wall;
the second position comprises a position located along a left ventricular wall; and
the comparing comprises a time domain analysis to determine a degree of synchrony between the wall motion at the first position and the wall motion at the second position.

29. The method of claim 23, wherein:
the first position comprises a position located in proximity to an apex of the heart;
the second position comprises a position located in proximity to a base of the heart; and
the comparing comprises a torsional analysis to determine a relative rotation between the first position and the second position.

30. The method of claim 23, wherein the at least one first cardiac pacing site comprises a left ventricular pacing site and a right ventricular pacing site.

31. The method of claim 23, further comprising:
applying cardiac pacing stimulation at an at least one second cardiac pacing site;
collecting a second set of paced heart wall motion data from the signal of each of the first and second electromagnetic receiver coils secured at the corresponding position;
comparing the set of non-paced heart wall motion data to the second set of paced heart wall motion data; and
determining, based on the comparing, whether to maintain pacing at the at least one first pacing site, or at the at least one second pacing site, or to apply pacing stimulation at an at least one third pacing site for collection of a third set of paced heart wall motion data.

32. A method for selecting at least one cardiac pacing site, the method comprising:
introducing a first elongate lead to a position along a right ventricular heart wall, the first lead including a first electromagnetic receiver coil;
coupling the first electromagnetic receiver coil at a position along the right ventricular heart wall;
inducing a first signal in the first electromagnetic receiver coil by generating a magnetic field, the first signal facilitating creation of a first virtual representation of a portion of the first lead to direct the first lead to a first position, and to track wall motion at the position along the right ventricular heart wall;
introducing a second elongate lead to a position along a left ventricular heart wall, the second lead including a second electromagnetic receiver coil;
coupling the second electromagnetic receiver coil at the position along the left ventricular heart wall;
inducing a second signal in the second electromagnetic receiver coil by generating a magnetic field, the second signal facilitating creation of a second virtual representation of a portion of the second lead to direct the second lead to a second position, and to track wall motion at the position along the left ventricular heart wall;
storing a set of non-paced heart wall motion data from the signal of each of the electromagnetic receiver coils secured at the corresponding position;
applying cardiac pacing stimulation at an at least one first cardiac pacing site;
collecting a first set of paced heart wall motion data from the signal of each of the electromagnetic receiver coils secured at the corresponding position;
comparing the set of non-paced heart wall motion data to the first set of paced heart wall motion data;
determining, based on the comparing, whether to maintain pacing at the at least one first cardiac pacing site or to apply pacing stimulation at a second cardiac pacing site for collection of a second set of paced heart wall motion data;
generating notification data which indicates that the first cardiac pacing site being one of an optimal pacing site and a non-optimal pacing site.

33. A computer-readable medium having stored thereon at least one instruction that, when executed by a computer, causes the computer to perform:
introducing a first elongate lead to a position along a right ventricular heart wall, the first lead including a first electromagnetic receiver coil;
coupling the first electromagnetic receiver coil at a position along the right ventricular heart wall;
inducing a first signal in the first electromagnetic receiver coil by generating a magnetic field, the first signal facilitating creation of a first virtual representation of a portion of the first lead to direct the first lead to a first position, and to track wall motion at the position along the right ventricular heart wall;
introducing a second elongate lead to a position along a left ventricular heart wall, the second lead including a second electromagnetic receiver coil;
coupling the second electromagnetic receiver coil at the position along the left ventricular heart wall;
inducing a second signal in the second electromagnetic receiver coil by generating a magnetic field, the second signal facilitating creation of a second virtual representation of a portion of the second lead to direct the second lead to a second position, and to track wall motion at the position along the left ventricular heart wall;
storing a set of non-paced heart wall motion data from the signal of each of the electromagnetic receiver coils secured at the corresponding position;
applying cardiac pacing stimulation at an at least one first cardiac pacing site;
collecting a first set of paced heart wall motion data from the signal of each of the electromagnetic receiver coils secured at the corresponding position;
comparing the set of non-paced heart wall motion data to the first set of paced heart wall motion data;
determining, based on the comparing, whether to maintain pacing at the at least one first cardiac pacing site or to apply pacing stimulation at a second cardiac pacing site for collection of a second set of paced heart wall motion data; and
generating notification data which indicates that the first cardiac pacing site comprises one of: an optimal pacing site and a non-optimal pacing site.

34. The computer readable medium of claim 33, wherein the optimal pacing site corresponds to a maximum difference in a voltage of the signal induced in the first and second electromagnetic receiver coils relative to a voltage reference.

35. The computer readable medium of claim 34, wherein the voltage reference comprises ground.
